(19) United States
(12) Patent Application Publication
(10) Pub. No.: US 2014/0221764 A1
(43) Pub. Date: Aug. 7, 2014

(54) STAT CLOCK FOR PATIENT SUPPORT APPARATUS

(71) Applicant: Hill-Rom Services, Inc., Batesville, IN (US)

(72) Inventors: William G. PITTENGER, Aurora, IN (US); Michael M. FRONDORF, Lakeside Park, KY (US)

(21) Appl. No.: 14/167,162

(22) Filed: Jan. 29, 2014

Related U.S. Application Data
(60) Provisional application No. 61/759,090, filed on Jan. 31, 2013.

(51) Int. Cl. A61G 7/018; A61B 19/00

(52) U.S. Cl. CPC A61G 7/018; A61B 19/5202
USPC 600/249; 128/845

(57) ABSTRACT

A patient support apparatus a frame, an emergency switch, and a controller. The frame is movable from an operating position to one of a plurality of emergency switches as a result of engagement of the emergency switch. The controller is configured to determine a time interval in which the frame is in one of the plurality of emergency positions as a result of engagement of the emergency switch.

### CROSS-REFERENCE TO RELATED U.S. PATENT APPLICATION

This present application claims priority under 35 U.S.C. §119(e) to U.S. Provisional Patent Application Ser. No. 61/759,090, entitled “STAT CLOCK FOR PATIENT SUPPORT APPARATUS,” which was filed on Jan. 31, 2013, the entirety of which is hereby incorporated by reference.

### BACKGROUND

The present disclosure is related to a patient support apparatus configurable in an operating and an emergency position, and in particular to a patient support apparatus including an emergency switch that causes the patient support apparatus to move toward the emergency position. More particularly, the present disclosure relates to operating a stat clock to measure an amount of time the patient support apparatus is in the emergency position.

During a medical emergency, a patient support apparatus is arranged in an appropriate emergency position. In one example, a patient experiences a medical emergency where Cardiopulmonary Resuscitation (CPR) is needed. As a result, the caregiver responding to the medical emergency arranges the patient support apparatus in a CPR position in which a patient support deck included in the patient support is in a substantially horizontal position. The caregiver may desire to know how long the emergency condition has been occurring to evaluate whether additional medical care is required, when additional medical care should be provided, and whether ongoing efforts to provide medical care should be stopped.

Tracking the time period in which the medical emergency exists may also be difficult as the primary goal during the medical emergency is to provide appropriate medical care to the patient. Caregivers, after the medical emergency has ended, enter the start time, end time, and time interval into the patient's medical history. As a result of the medical emergency often being hectic and stressful, both the start and end time of the medical emergency may be estimated. Thus, recording the time period into a patient's medical history may be susceptible to errors or may be forgotten altogether.

### SUMMARY

Additional features, which alone or in combination with any other feature(s), including those listed above and those listed in the claims may comprise patentable subject matter and will become apparent to those skilled in the art upon consideration of the following detailed description of illustrative embodiments exemplifying the best mode of carrying out the embodiments as presently perceived.

According to one aspect of the present disclosure, a patient support apparatus comprises a frame, a first emergency input indicative of an emergency condition, and a controller. The frame is movable between an operating position and a first emergency position. The first emergency input is coupled to the frame to cause the frame to move toward the first emergency position in response to engagement of the first emergency input. The controller is coupled to the first emergency input to detect engagement of the first emergency input. Once the controller detects engagement of the first emergency input, the controller makes an entry into a patient's electronic medical record of the engagement of the first emergency input.

The controller may be configured to transmit the entry via a communication network to an electronic database for inclusion in the patient's electronic medical record. The controller may include memory and the controller may store the entry in the memory. The controller may be further configured to transmit the entry stored in the memory via a communication network to an electronic database for inclusion in the patient's electronic medical record.

The patient support apparatus may further comprise a second input that may be indicative of an end to the emergency condition. The controller may be further configured to detect engagement of the second input and may add to the entry engagement of the second input. The patient support apparatus may further comprise a graphical user interface that may be coupled to the frame. The controller may be coupled to the graphical user interface to cause the graphical user interface to display a visual indicator. The visual indicator may communicate that the emergency condition is occurring in response to engagement of the first emergency input.

The patient support apparatus may further comprise an emergency light. The emergency light may be coupled to the controller and may be configured to provide light in response to engagement of the first emergency input. The emergency light may be located in a patient room with the patient support apparatus. The patient support apparatus may be located in the patient room and the emergency light may be located outside the patient room.

The patient support apparatus may further comprise an emergency light. The emergency light may be coupled to the controller and may be configured to provide light in response to engagement of the first emergency input. The controller may be further configured to send a priority nurse call to a hospital information system via a communication network in response to engagement of the first emergency input.

The first emergency input may be a CPR handle. The CPR handle may be coupled to the frame to move relative to the frame between an engaged position and a disengaged position. The engaged position may cause the frame to move toward the first emergency position. The disengaged position may allow the frame to remain in the operating position. The first emergency position may be a CPR position.

According to another aspect of the present disclosure, a patient support apparatus comprises a frame and a first emergency input indicative of an emergency condition. The frame is movable between an operating position and a first emergency position. The first emergency input is coupled to the frame to cause the frame to move toward the emergency position in response to engagement of the first emergency input. The patient support apparatus further comprises a second input and a controller. The second input is indicative of an end to the emergency condition. The controller is coupled to the first emergency input and the second input to detect engagement of the first emergency input and make an entry into a patient's medical record of the engagement of the first emergency input. The controller is further coupled to the first emergency input and the second input to detect engagement of the second input and add to the entry engagement of the second input. The controller is further coupled to the first emergency input and the second input to add to the entry a date and a time in which the first emergency input was engaged and the second input was engaged. The controller is further coupled to the first emergency input and the second input to communicate the entry via a communication network to an electronic database for inclusion in the patient's electronic medical record.

The patient support apparatus may further comprise a graphical user interface. The graphical user interface may be coupled to the frame. The controller may be coupled to the graphical user interface to cause the graphical user interface to display a visual indicator. The visual indicator may communicate that the emergency condition is occurring in response to engagement of the first emergency input. The controller may further be coupled to the graphical user interface to cause the graphical user interface to display a button which is the second input.

According to another aspect of the present disclosure, a patient support apparatus comprises a frame and a first emergency input indicative of an emergency condition. The frame is movable between an operating position and a first emergency position. The first emergency input is coupled to the frame to cause the frame to move toward the emergency position in response to engagement of the first emergency input. The patient support apparatus further comprises a controller. The controller is coupled to the first emergency input to detect engagement of the first emergency input and start a timer upon engagement of the first emergency input.

The controller may be configured to make an entry into a patient's medical record of the engagement of the first emergency input. The controller may be further configured to communicate the entry via a communication network to an electronic database for inclusion in a patient's electronic medical record.

The patient support apparatus may further comprise a graphical user interface. The controller may be coupled to the graphical user interface to cause the timer to be displayed upon engagement of the first emergency input. The patient support apparatus may further comprise a second input. The second input may be indicative of an end to the emergency condition.

The controller may be further configured to cause a real-time clock to be displayed on the graphical user interface. The real-time clock may be displayed on the graphical user interface at the same time the timer is displayed. The controller may be further configured to detect engagement of the second input and add to the entry engagement of the second input. The controller may be further configured to calculate a time interval in response to detecting the second input and add the time interval to the entry. The controller may be further configured to communicate the entry via a communication network to an electronic database for inclusion in the patient's electronic medical record. The controller may be further configured to add a time and a date to the entry in which the first emergency input was detected. The controller may be further configured to add a time and a date to the entry in which the second input was detected.

The controller may be coupled to the graphical user interface to cause the time interval to be displayed upon engagement of the second input. The second input may be a button that is displayed on the graphical user interface. The second input may be detected by the controller in response to movement of the frame from the first emergency position to the operating position.

The first emergency input may be a CPR handle. The CPR handle may be coupled to the frame to move relative to the frame between an engaged position and a disengaged position. The engaged position may cause the frame to move toward the first emergency position. The disengaged position may allow the frame to remain in the operating position. The first emergency position may be a CPR position.

The controller may be further configured to send a priority nurse call to a hospital information system via a communication network in response to engagement of the first emergency input. The patient support apparatus may further comprise an emergency light. The emergency light may be coupled to the controller and configured to provide light in response to engagement of the first emergency input.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a partial perspective view of a patient's room located in a hospital showing a patient support apparatus in accordance with the present disclosure;

FIG. 2 is a diagrammatic view showing the patient support apparatus arranged in a first emergency position associated with a CPR position, a second emergency position associated with a Trendelenburg position, and a third emergency position associated with a Reverse Trendelenburg position;

FIG. 3 is a diagrammatic view showing local communications associated with the patient support apparatus and hospital communications associated with a hospital network;

FIGS. 4A-4C are a series of diagrammatic views showing a process for monitoring a patient support apparatus for arrangement in one of the emergency positions and reporting the time period the patient support apparatus is in one of the emergency positions to the hospital network for entry into the patient's electronic medical records;

FIG. 5 is a diagrammatic view of a controller included in the patient support apparatus; and

FIG. 6 is a perspective view of a user interface included in the patient support apparatus.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

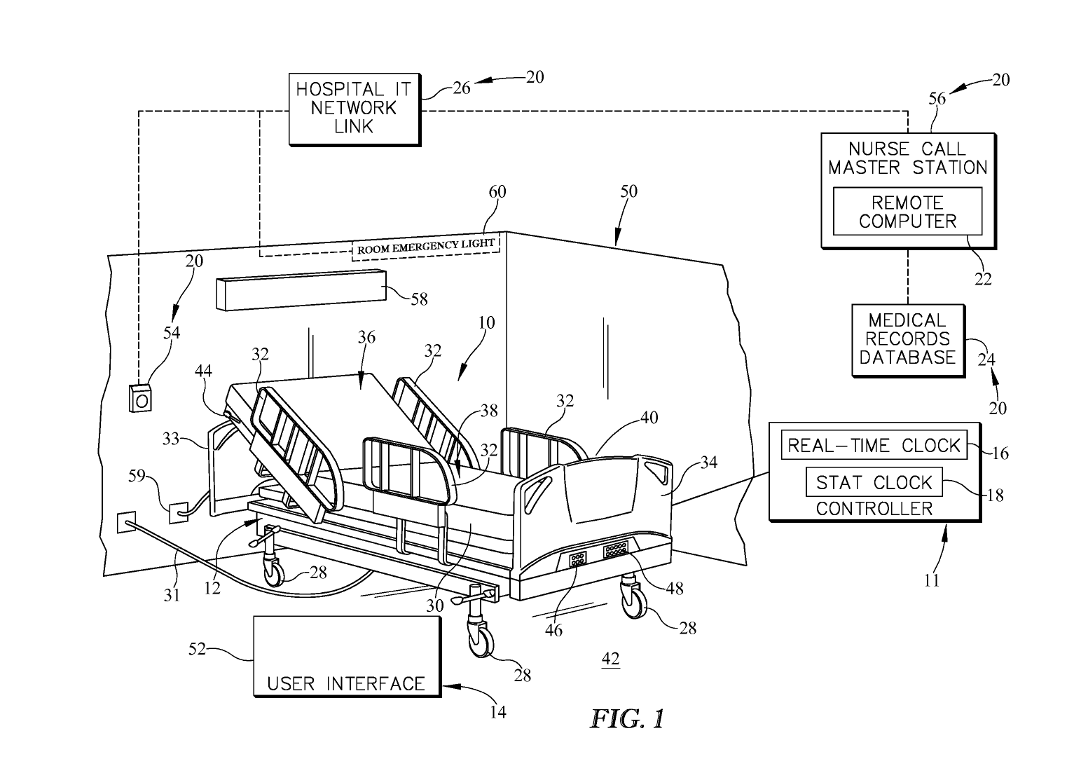

Referring to FIG. 1, a patient support apparatus is embodied as a hospital bed 10. The bed 10 includes a frame 12 that is movable between an operating position and a plurality of emergency positions in response to engagement of one or more emergency switches, also called emergency inputs. Each emergency switch is indicative of an emergency condition associated with a patient or with the bed 10. The bed 10 further includes a controller 11 that is configured to detect the engagement of each emergency switch and make an entry into a patient's medical record reflecting the engagement of each emergency switch. The controller 11 is coupled to a healthcare communication network 20 through which the controller 11 accesses an electronic database 24 containing the patient's medical record to make the entry. The controller 11 is also configured to detect engagement of a second input indicative of an end to the emergency condition. The controller 11 is also configured to add the time and date in which the emergency switch and the second input were engaged to the entry.

Bed 10 is coupled to the healthcare communication system 20, also called the communication network, that includes a computer 22 providing access to a medical records database 24 maintained by the hospital as shown in FIG. 1. Medical records database 24 includes a plurality of electronic history files that are maintained for patients that receive care at the hospital. When an emergency condition is associated with bed 10, such as a patient resting on bed 10 requiring an emergency procedure, bed 10 is placed in one of the emergency positions by engaging one of the emergency switches. The engagement of the emergency switches is detected by the controller 11 and indicated in the entry made in the patient's medical record as discussed above. The engagement of the emergency switch also initiates a timer embodied as a stat clock 18 in FIG. 1. The stat clock 18 is included in the controller 11 and configured to measure the time elapsing immediately after the controller 11 detects the engagement of one of the emergency switches. The stat clock 18 is also configured to calculate a time interval that starts with the engagement of the emergency switch and ends with the engagement of the second input. The controller 11 communicates the time interval to the computer 22 through a hospital network 26 included in the healthcare communication system 20. The time interval is added to the entry and may be accepted into an official record of care administered to the patient.

Referring again to FIG. 1, the frame 12 included in the bed 10 is supported by wheels or casters 28. The bed 10 includes a mattress 30 supported by the frame 12, a number of siderails 32, a footboard 34, and a headboard 33. The bed 10 also includes multiple support sections such as a head support section 36, a midsection support section 38, and a foot support section 40 as shown in FIG. 1. The frame 12 is adjustably operable to place the bed support sections in multiple positions relative to one another.

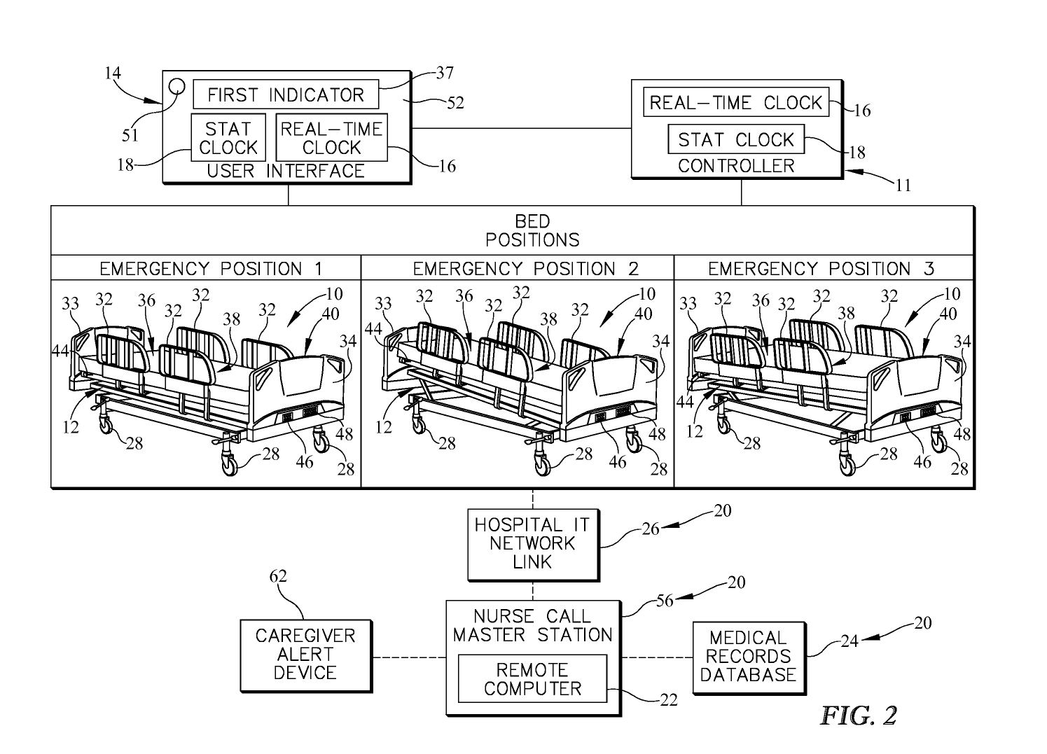

In one example, the frame 12 may be adjusted to place the bed 10 in the Trendelenburg position in which the foot support section 40 is inclined about 15-30 degrees above the head support section 36 and the midsection support section 38 relative to ground 42 on which the bed 10 rests. A patient resting on the bed 10 while the bed 10 is inclined in the Trendelenburg position receives leg and foot support from the foot support section 40 at a first distance above ground 42, midsection support from the midsection support section 38 at a second distance above ground 42, and head support from the head support section 36 at a third distance above ground 42. As shown in FIG. 2, the first distance is greater than the second distance and the second distance is greater than the third distance when the frame 12 is in the Trendelenburg position.

In another example, the frame 12 may be adjusted to place the bed 10 in the reverse Trendelenburg position in which the head support section 36 and the midsection support section 38 are inclined about 15-30 degrees above the foot support section 40 relative to ground 42. The patient resting on the bed 10 while the bed 10 is inclined in the reverse Trendelenburg position receives head support from the head support section 36 at a first distance above ground 42, midsection support from the midsection support section 38 at a second distance above ground 42, and leg and foot support from the foot support section 40 at a third distance above ground 42. As shown in FIG. 2, the first distance is greater than the second distance and the second distance is greater than the third distance when the frame 12 is in the reverse Trendelenburg position.

In addition to both the Trendelenburg and reverse Trendelenburg positions, the frame 12 may be adjusted to place the bed 10 in a CPR position in which the head support section 36, the midsection support section 38, and the foot support section 40 lie substantially parallel to one another and ground 42 as shown in FIG. 2. The patient resting on the bed 10 while the bed 10 is in the CPR position receives head support from the head support section 36, midsection support from the midsection support section 38, and leg and foot support from the foot support section 40 at substantially the same distance above ground 42 as shown in FIG. 2.

For the purposes of the present disclosure, the Trendelenburg position, the reverse Trendelenburg position, and the CPR position are considered to be emergency positions associated with the emergency switches. The frame 12 is adjustably operable to place the bed 10 in the operating position in which caregivers do not prepare to administer emergency care to the patient. An example of the operating position is shown in FIG. 1.

Referring again to FIG. 1, the plurality of emergency switches or emergency inputs included in the bed 10 includes a CPR handle 44, a Trendelenburg pedal 46, and a reverse Trendelenburg pedal 48. The CPR handle 44 is coupled to the frame 12 along the head support section 36 of the bed 10 and is accessible to a caregiver providing care for the patient resting on the bed 10 within a patient's room 50. The Trendelenburg pedal 46 is coupled to the frame 12 along foot support section 40 of the bed 10. The Reverse Trendelenburg pedal 48 is coupled to the frame 12 along foot support section 40 of the bed 10 similar to the Trendelenburg pedal 46.

As suggested in FIG. 2, the CPR handle 44 is coupled to the frame 12 and may be used to adjust the frame 12 to move the bed 10 toward the CPR position. The CPR handle 44 is movable relative to the frame 12 and may be engaged to move the bed 10 toward the CPR position until the bed 10 is placed in the CPR position. The CPR handle 44 may also be disengaged to allow the frame 12 to remain in the operating position. The CPR handle 44 is coupled to the controller 11 and provides user input thereto, such as operating input associated with the operating position and emergency input associated with the CPR position. The CPR handle 44 is shown engaged in FIG. 2 and disengaged in FIG. 1.

As suggested in FIG. 2, the Trendelenburg pedal 46 is coupled to the frame 12 and may be used to adjust the frame 12 to move the bed 10 toward the Trendelenburg position. The Trendelenburg pedal 46 is movable relative to the frame 12 and may be engaged to move the bed 10 toward the Trendelenburg position until the bed 10 is placed in the Trendelenburg position. The Trendelenburg pedal 46 may also be disengaged to allow the frame 12 to remain in the operating position. The Trendelenburg pedal 46 is coupled to the controller 11 and provides user input thereto, such as operating input associated with the operating position and emergency input associated with the Trendelenburg position. The Trendelenburg pedal 46 is shown engaged in FIG. 2 and disengaged in FIG. 1.

As suggested in FIG. 2, the Reverse Trendelenburg pedal 48 may be used to adjust the frame 12 to move the bed 10 toward the Reverse Trendelenburg position. The Reverse Trendelenburg pedal 48 is movable relative to the frame 12 and may be engaged to move the bed 10 toward the Reverse Trendelenburg position until the bed 10 is placed in the Reverse Trendelenburg position. The Reverse Trendelenburg pedal 48 may also be disengaged to allow the frame 12 to remain in the operating position. The Reverse Trendelenburg pedal 48 is coupled to the controller 11 and provides user input thereto, such as operating input associated with the operating position and emergency N1-37302 input associated with the Reverse Trendelenburg position. The Reverse Trendelenburg pedal 48 is shown engaged in FIG. 2 and disengaged in FIG. 1.

In some embodiments, the bed 10 may include a number of electronically controlled functions. For example, the bed 10 may include a patient input-output device capable of receiving and processing electrical input from a number of manually operable switches coupled to the patient input-output device. The patient input-output device is used to enable the patient to activate and deactivate certain functions when the patient is positioned on the bed 10. Such functions might include adjusting the frame 12 to place the bed 10 in the CPR position, the Trendelenburg or reverse Trendelenburg positions, placing a call to a hospital communication system, such as a nurse call system, or turning on lighting or other room devices. In some embodiments, the bed 10 includes a caregiver input-output device capable of receiving and processing electrical inputs from a number of manually operable switches coupled to the caregiver input-output device. Similar to the patient input-output device, the caregiver input-output device enables the caregiver to configure, activate, and/or deactivate certain of the electronically controlled bed functions.

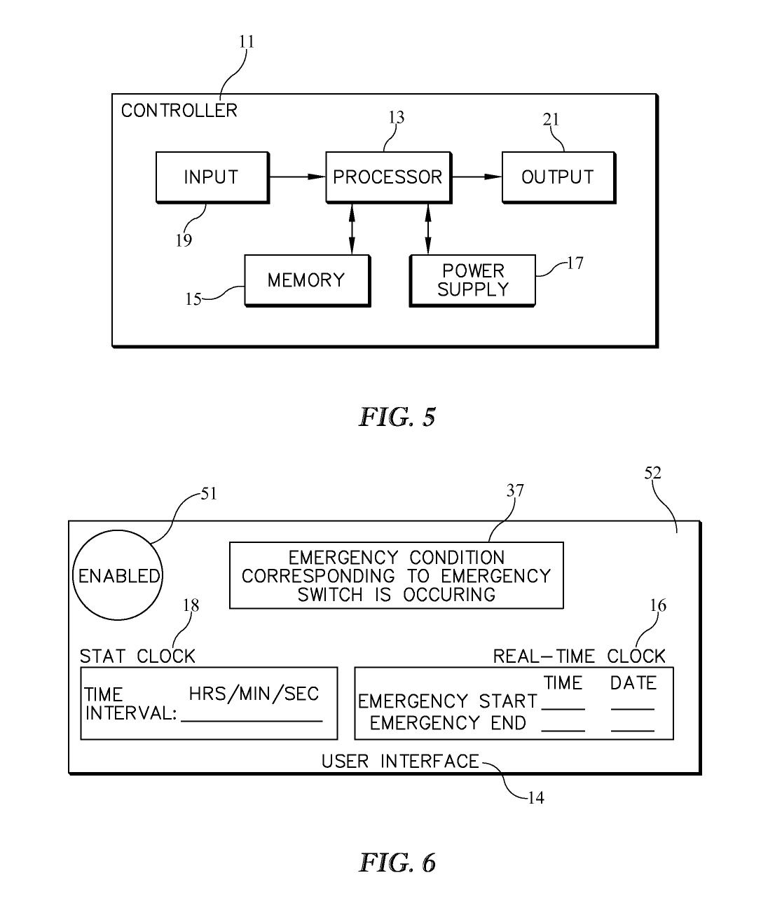

The controller 11, as shown in FIG. 5, manages electronically controlled functions associated with the bed 10. The controller 11 includes a processor 13 and memory 15. The processor 13 receives power from a power supply 17. The power supply 17 may be located external to bed 10 within the patient's room 50 or in the bed 10. The processor 13 is configured to execute instructions stored in memory 15. Memory 15 may be used to store the entry made in the patient's medical record. The controller 11 is configured to receive input signals from one or more inputs 19, such as the CPR handle 44, the Trendelenburg pedal 46, or the Reverse Trendelenburg pedal 48, which are coupled to the controller 11. The controller 11 is also configured to transmit output signals to one or more outputs 21, such as the healthcare communication system 20, which is coupled to the controller 11. In one example, the controller 11 may transmit the entry stored in memory 15 to the medical records database 24 via the hospital network 26 for inclusion in the patient's medical record. The controller 11 may be contained within a housing that is mountable to a fixed location on the bed 10. The housing may also contain electrical circuitry included with the controller 11. The controller 11 may be coupled to the frame 12, one of the siderails 32, the headboard 33, or the footboard 34, or other suitable location.

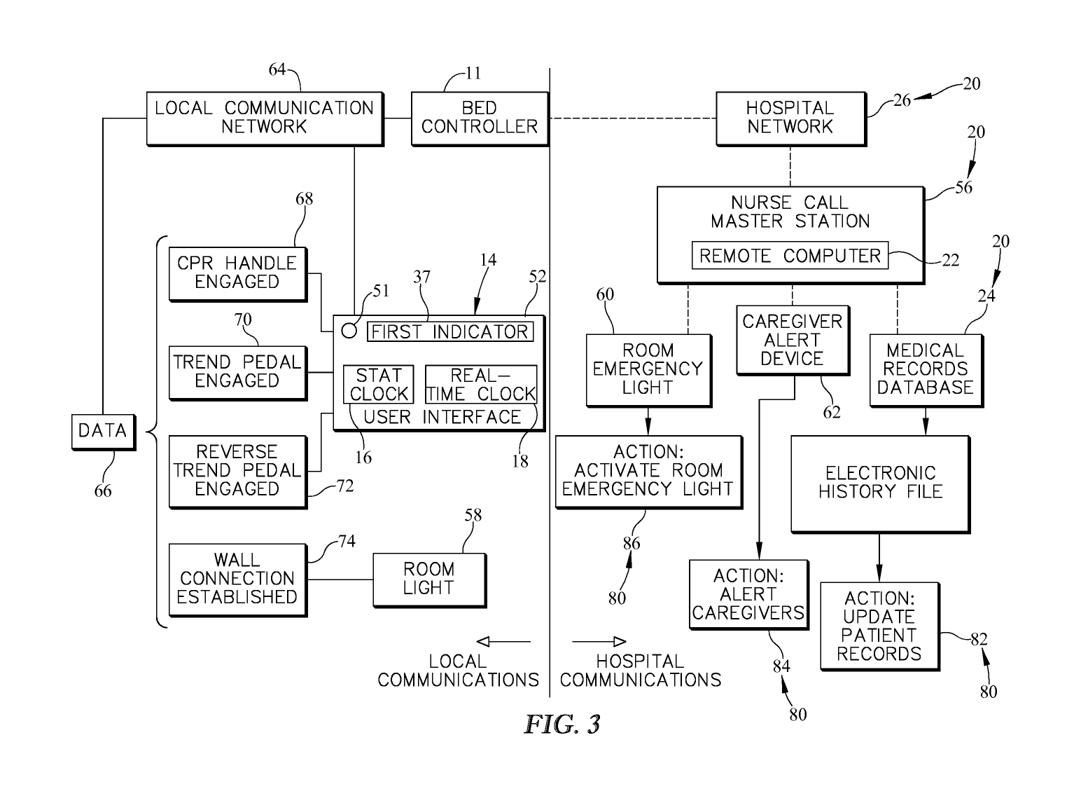

In some embodiments, the controller 11 receives electrical inputs from other bed modules or devices, such as the patient input-output device or the caregiver input-output device, via a bed network. In one example, the bed network is be an electronic network configured according to a CAN (Controller Area Network) or Echelon protocol, or any other suitable bed network communications protocol. For the purposes of the present disclosure, the controller 11 is coupled to each of the emergency switches through a bed network such as the local communication network 64 as shown in FIG. 3.

As shown in FIG. 1, the user interface 14 illustratively embodies a caregiver input-output device associated with the bed 10. The user interface 14 is configured to communicate with the controller 11 of the bed 10 and may be coupled to the bed 10 or physically detached from the bed 10 within the patient's room 50. The user interface 14 includes a display 52 which enables the caregiver to configure, activate, and/or deactivate certain of the electronically controlled bed functions or the room devices. The display 52 may be configured to display visual indications viewable by the caregiver or other persons in the patient's room 50 in response to commands, instructions, or user input received from the controller 11. The display 52 may also be configured to emit audible indications in response to commands, instructions, or user input received from the controller 11.

In one example, the user interface 14 displays a first indicator 37 communicating that one of the emergency switches is engaged and that the emergency condition corresponding to the emergency switch is occurring as shown in FIG. 6 and suggested in FIG. 2. In another example, the user interface 14 displays the first indicator 37 on the display 52 and a second input 51 as shown in FIG. 6.

In some embodiments, the second input 51 is a button that can be enabled and disabled, also called engaged and disengaged, respectively, by hospital personnel having access to the user interface 14. In one example, the second input 51 is enabled to indicate the end of the emergency condition associated with the first indicator 37. In some examples, the second input 51 is disabled to indicate that the emergency condition signified by the first indicator 37 has not yet ended. The controller 11 detects that the second input 51 is enabled in response to movement of the frame 12 from one of the emergency positions to the operating position. As previously stated, the second input 51 is enabled to end the time interval calculated by the stat clock 18 that starts with the engagement of the emergency switch. The time and date at which the second input 51 is enabled and disabled is included in the entry made by the controller 11.

As shown in FIG. 1, the controller 11 includes a real-time clock 16 and the stat clock 18. The controller 11 is coupled to the user interface 14 so that the real-time clock 16 and the stat clock 18 may be displayed on the display 52 as shown in FIG. 6. Both the real-time clock 16 and the stat clock 18 are displayed on the display 52 once the controller 11 detects that one of the emergency switches is engaged as shown in FIG. 6 and suggested in FIG. 2. The real-time clock 16 and the stat clock 18 may be displayed on the display 52 simultaneously or at different times. In addition, the time interval calculated by the stat clock 18 and commencing with the engagement of one of the emergency switches and ending with the engagement of the second input 51 is displayed on the display 52 once the controller 11 detects that the second input 51 is engaged as shown in FIG. 6. The information displayed on the display 52, including the real-time clock 16, the stat clock 18, and the interval calculated by the stat clock 18, may be communicated from the bed 10 to the medical records database 24 through the hospital network 26 for inclusion in the patient's medical record. The information is included in the entry made by the controller 11.

Electrical communications that originate at the bed 10, through either the controller 11 or the user interface 14, may be communicated to the healthcare communication system 20 as shown in FIG. 1. The healthcare communication system 20 may include a number of stations or consoles that cooperate with various computing devices, networks, and supporting equipment or services to enable caregivers and other staff to receive, view, manage, and route, output or respond to electrical and wireless signals from a variety of communication, call, monitoring, detecting and/or signaling devices located in the patient's room 50. The healthcare communication system 20 includes a nurse call user station 54, the hospital network 26, and a nurse call master station 56 as shown in FIG. 1.

The nurse call user station 54 provides a communication interface usable by the caregiver or patient to communicate with other hospital personnel located in areas of the hospital outside of the patient's room 50. The nurse call user station 54 may include a computer or computing device that has a display screen, voice communication capabilities, and one or more input devices (such as a keyboard, touch screen, mouse, switch, button, knob, or the like) configured to control the operation of the healthcare communication system 20. Voice communication capabilities may be provided by an integrated microphone and speaker and/or a telephone handset included in the nurse call user station 54. As shown in FIG. 1, the nurse call user station 54 communicates with the nurse call master station 56 through the hospital network 26. The nurse call user station 54 is coupled to the controller 11 of the bed 10 through a wired connection 31 as shown in FIG. 1.

The nurse call master station 56, also referred to as a hospital information system, includes the computer 22 that is configured to provide access to medical records database 24 as shown in FIG. 1. The computer 22 is configured to provide an interface to the healthcare communication system 20 for a user, such as a nurse or other hospital personnel. The computer 22 includes an output device, such as a visual display or speaker that is used to notify or communicate calls (such as nurse calls) and/or other information to the user. The computer 22 may also include an input device such as a touchscreen, keypad or keyboard, microphone, telephone handset, push button, switch, dial, lever, or the like, to enable the user to place and/or respond to calls or other information. The computer 22 includes circuitry configured for connection to the hospital network 26.

The hospital network 26 is configured to enable caregivers and patients located in hospital rooms such as the patient's room 50 to communicate with hospital personnel located in other areas of the hospital. Caregiver or patient communications originating from the patient's room 50 may be communicated to the nurse call master station 56 through the hospital network 26 as shown in FIG. 1. Similarly, caregiver or other hospital personnel communications originating at the nurse call master station 56 may be communicated to patient's room 50 through hospital network 26 as shown in FIG. 1. In one example, controller 11 is configured to send a first priority nurse call to nurse call master station 56 through the hospital network 26 in response to detecting the engagement of one of the emergency switches indicating the corresponding emergency condition associated with the bed 10. In another example, the controller 11 is configured to send a second priority nurse call to the nurse call master station 56 through the hospital network 26 indicating that the second input 51 is enabled and that the emergency condition associated with the bed 10 has been resolved.

As shown in FIG. 1, the patient's room 50 includes a room light 58 that is coupled to one of the walls defining the interior space provided within the patient's room 50. The room light 58 is arranged above the bed 10 as an overbed light as shown in FIG. 1. The room light 58 is configured to provide light when activated to illuminate a portion of the patient's room 50. For the purposes of this disclosure, the room light 58 may be arranged anywhere within the patient's room 50 and may serve as a night light or reading light rather than an overbed light. In some embodiments, the patient's room 50 includes one or more room devices, such as a night/reading light, a television or entertainment system, or other lighting devices. The room devices, in some examples, are coupled to the bed 10 and in other examples spaced apart from the bed 10 within the patient's room 50.

The room light 58 is coupled to the controller 11 of the bed 10 through the interface 59 as shown in FIG. 1. The interface 59 is disposed on one of the walls included in the patient's room 50 and lies proximate to the headboard 33 below the room light 58. The interface 59 enables the functionality of the room light 58 to be controlled by the controller 11 when a connection is established between the bed 10 and the interface 59. For instance, engagement of the CPR handle 44, the Trendelenburg pedal 46, or the Reverse Trendelenburg pedal 48 may be used to start the stat clock 18 (the timer) and activate the room light 58 through the interface 59. Conversely, enablement of the second input 51 may be used to stop the stat clock 18 and de-activate the room light 58 through the interface 59.

In some embodiments, an emergency light 60 is located outside of the patient's room 50 in a corridor or hallway provided within the hospital as suggested in FIG. 1. In another example, the emergency light 60 is located within the patient's room 50. The emergency light 60 is coupled to the controller 11 through the local communication network 64 as shown in FIG. 3. The emergency light 60 is configured to activate to alert caregivers or other hospital personnel to emergency conditions associated with the patient resting on the bed 10 within the patient's room 50 following the engagement of one of the emergency switches. For instance, the emergency light 60 may be activated once the controller 11 detects that the CPR handle 44 is engaged, the Trendelenburg pedal 46 is engaged, or the Reverse Trendelenburg pedal 48 is engaged. Conversely, de-activation of the emergency light 60 may provide an indication to caregivers or other hospital personnel that the emergency condition has been resolved. For instance, the emergency light 60 may be de-activated once the controller 11 detects that the second input 51 is enabled.

Referring again to FIG. 1, the emergency light 60 is configured to communicate with the nurse call master station 56 through the hospital network 26. Activation of the emergency light 60 communicated to hospital personnel stationed at the nurse call master station 56 through the hospital network 26 to provide an alert that the controller 11 has detected engagement of one of the emergency switches. De-activation of the emergency light 60 is communicated to hospital personnel stationed at the nurse call master station 56 through the hospital network 26 to provide an indication that the controller 11 has detected engagement of the second input 51.

Referring to FIG. 2, the bed 10 is shown in each of the emergency positions. Emergency position 1 corresponds to the CPR position, emergency position 2 corresponds to the reverse Trendelenburg position, and emergency position 3 corresponds to the Trendelenburg position as shown in FIG. 2. The stat clock 18 is configured to calculate the interval beginning once the controller 11 detects that one of the emergency switches is engaged to place the bed 10 in one of the emergency positions and ending once the controller detects that the second input 51 is enabled. In some embodiments, the second input 51 is engaged once the frame 12 is moved from any of the emergency positions to the operating position.

As each of the emergency switches is shown in the engaged position in emergency positions 1-3, the first indicator 37 is displayed on the display 52 of the user interface 14 as shown in FIG. 6. The second input 51 is also displayed on the display 52 in response to the controller 11 making the entry in the patient's medical record (using the healthcare communication system 20). The stat clock 18 is also displayed on the display 52 and tracks the time elapsing since the detected engagement of the emergency switch. The real-time clock 16 is simultaneously displayed with the stat clock 18 and identifies the time and date at which the first indicator 37 appeared on the display 52 as well as the time and date at which the second input 51 is enabled. The second input 51 is enabled in FIG. 6 so that the stat clock 18 counts the time interval and displays the time interval on the display 52.

The time interval calculated using the stat clock 18 may be added to the entry made by the controller 11 and communicated to the nurse call master station 56 through the hospital network 26 as shown in FIGS. 2 and 3. The user uses the computer 22 included in the nurse call master station 56 to access the medical records database 24 to retrieve patient information for the patient resting on the bed 10. Such patient information optionally include the entry made by the controller 11, patient name, date of birth, prescription information, biometric data including blood type, fingerprints, or retinal scan data, and the tracking of administered medication and treatment over time. Patient information for the patient resting on the bed 10 is captured in the electronic history file maintained for the patient and stored on the medical records database 24.

The entry made by the controller 11 includes the time and date at which the emergency switch and the second input 51 is engaged/enabled as suggested in FIG. 6. In dome embodiments, the user verifies the correctness of the electronically recorded time interval by providing an electronic signature using the computer 22. The electronic signature provides evidence of compliance with a hospital protocol for administering medical care and delivering treatment.

The healthcare communication system 20 serves as a network that is included within a hospital telecommunications infrastructure and is configured to facilitate communication among a variety of telecommunication devices. Such telecommunication devices include the caregiver alert devices 62 as shown in FIG. 2. The caregiver alert devices 62 include analog and digital devices, fixed telephones and mobile or cellular devices, personal data assistants (PDAs), pagers, and the like.

As shown in FIG. 2, the caregiver alert devices 62 are configured to communicate with the nurse call master station 56 through the hospital network 26. In one example, the caregiver alert devices 62 provide alerts to caregivers signifying emergency conditions associated with the engagement of any one of the emergency switches. In another example, the caregiver alert devices provide indications to caregivers signifying that the second input 51 is enabled and that the emergency condition associated with the emergency switch is resolved.

In some embodiments, the caregiver alert devices 62 also provide access to the medical records database 24. The time interval calculated by the stat clock 18 and included in the entry is by the controller 11 may be communicated to the nurse call master station 56 and thereafter to the caregiver alert devices 62 through the hospital network 26 as suggested in FIG. 2. The caregivers use the devices 62 to verify the correctness of the time interval in the patient's electronic history file (medical record) by providing an electronic signature. The electronic signature made using the devices 62 provides evidence of compliance with a hospital protocol for administering medical care and delivering treatment.

Referring to FIG. 3, communications originating at the bed 10 or associated with devices located within the patient's room 50 (described as local communications) are shown separately from communications using the healthcare communications system 20 (described as hospital communications). Local communications are confined largely within the patient's room 50 whereas hospital communications may take place outside of the patient's room 50 within the hospital.

Local communications are achieved through the use of the local communication network 64 as shown in FIG. 3. The local communication network 64 is configured to communicate bed data 66 originating from the bed 10 and from devices located in the patient's room 50 to the controller 11 as shown in FIG. 3.

Bed data 66 includes data generated using manually-operable mechanical switches such as the CPR handle 44, the Trendelenburg pedal 46, and the Reverse Trendelenburg pedal 48 that are coupled to the frame 12 and the user interface 14. Bed data 66 may also include, in some embodiments, data generated from movement of the CPR handle 44 in one direction to move the frame 12 toward the CPR position, movable in a second direction to move the frame 12 toward the Trendelenburg position. In yet other embodiments, bed data 66 may be generated as a result of the CPR handle 44 being engaged to cause the frame 12 to move toward one of the emergency positions. The CPR handle 44 may remain engaged to cause the frame 12 to move from emergency position to another emergency position. Bed data 66 further includes connectivity information associated with the bed 10 and the wall interface 59 provided in one of the walls of the patient's room 50. Examples of bed data 66 include a CPR handle engaged signal 68, a Trendelenburg pedal engaged signal 70, a reverse Trendelenburg pedal engaged signal 72, and a wall connection established signal 74 as shown in FIG. 3.

The CPR handle engaged signal 68 is generated by engaging the CPR handle 44 to move the frame 12 toward the CPR position shown in FIG. 2. The CPR handle engaged the signal 68 is communicated to the user interface 14 which prompts the first indicator 37 to be displayed on the display 52 of the user interface 14. As stated above and shown in FIG. 6, the stat clock 18, the real-time clock 16, and the second input 51 may be displayed on the display 52 in response to the engagement of the CPR handle 44. Once the second input 51 is enabled, the stat clock 18 calculates the time interval and displays the time interval as shown in FIG. 6. As suggested in FIG. 3, the time interval calculated by the stat clock 18 is communicated from the controller 11 to the user interface 14 through the local communication network 64.

The Trendelenburg pedal engaged signal 70 is generated by engaging the Trendelenburg pedal 46 to move the frame 12 toward the Trendelenburg position shown in FIG. 2. The Trendelenburg pedal engaged signal 70 is communicated to the user interface 14 which prompts the first indicator 37 to be displayed on the display 52 of the user interface 14. In the same manner described with respect to the CPR handle engaged signal 68, the time interval calculated by the stat clock 18 is communicated from the controller 11 to the user interface 14 through local communication network 64.

The Reverse Trendelenburg pedal engaged signal 72 is generated by engaging the reverse Trendelenburg pedal 48 to move the frame 12 toward the Reverse Trendelenburg position shown in FIG. 2. The Reverse Trendelenburg pedal engaged signal 72 is communicated to the user interface 14 which prompts the first indicator 37 to be displayed on the display 52 of the user interface 14. In the same manner described with respect to both the CPR handle engaged signal 68 and the Trendelenburg pedal engaged signal 70, the time interval calculated by the stat clock 18 is communicated from the controller 11 to the user interface 14 through the local communication network 64.

The wall connection established signal 74 is generated once a connection is established between the bed 10 and the interface 59. The connection between the bed 10 and the interface 59 is established to activate the room light 58 in response to the engagement of the CPR handle 44, the Trendelenburg pedal 46, or the reverse Trendelenburg pedal 48. The state of the room light 58 is communicated from the room light 58 to the controller 11 through the local communication network 64 as shown in FIG. 3.

The hospital communications take place as information is communicated from the controller 11 to locations outside of the patient's room 50 including the nurse call master station 56 and devices located outside of the patient's room 50 including the caregiver alert devices 62 as shown in FIG. 3. The hospital communications use the healthcare communication system 20 to communication information from the controller 11 to locations outside of the patient's room 50.

Bed data 66 including the CPR handle engaged signal 68, the Trendelenburg pedal engaged signal 70, the reverse Trendelenburg pedal engaged signal 72, and the wall connection established signal 74 is communicated from the controller 11 to the nurse call master station 56 through the hospital network 26 as shown in FIG. 3. Bed data 66 reaching the nurse call master station 56 initiates the performance of one or more operator tasks 80. As shown in FIG. 3, operator tasks 80 include update patient records task 82, alert caregivers task 84, and activate emergency light task 86.

The verify patient records task 82 includes the use of the computer 22 to access the medical records database 24 and update the patient's electronic history file in light of bed data 66 received at the nurse call master station 56 as shown in FIG. 3. The update patient records task 82 is performed by a caregiver, operator, or other hospital personnel. In some embodiments, the update patient records task 82 requires the nurse call master station 56 occupant to verify the time interval calculated by the stat clock 18 by signing electronically. The electronic signature provides evidence of compliance with the hospital protocol for administering medical care and delivering treatment.

The alert caregivers task 84 entails the transmission of an alert message or indication from the nurse call master station 56 to the caregiver alert devices 62 using the hospital network 26 as shown in FIG. 3. The alert message contains information concerning bed data 66 communicated from the controller 11 to the nurse call master station 56 such as the time interval calculated by the stat clock 18. As previously stated, caregivers use the devices 62 to verify the time interval in the patient's electronic history file by signing electronically.

The activate emergency light task 86 includes the activation of the emergency light 60 from the nurse call master station 56 as shown in FIG. 3. As previously stated, the activation of the emergency light 60 may coincide with the engagement of one of the emergency switches (and the calculating of the time interval using the stat clock 18) and de-activation of the emergency light 60 may indicate that the time interval has ended. Activation of the emergency light 60 may also alert caregivers or other hospital personnel to emergency conditions associated with the patient resting on the bed 10.

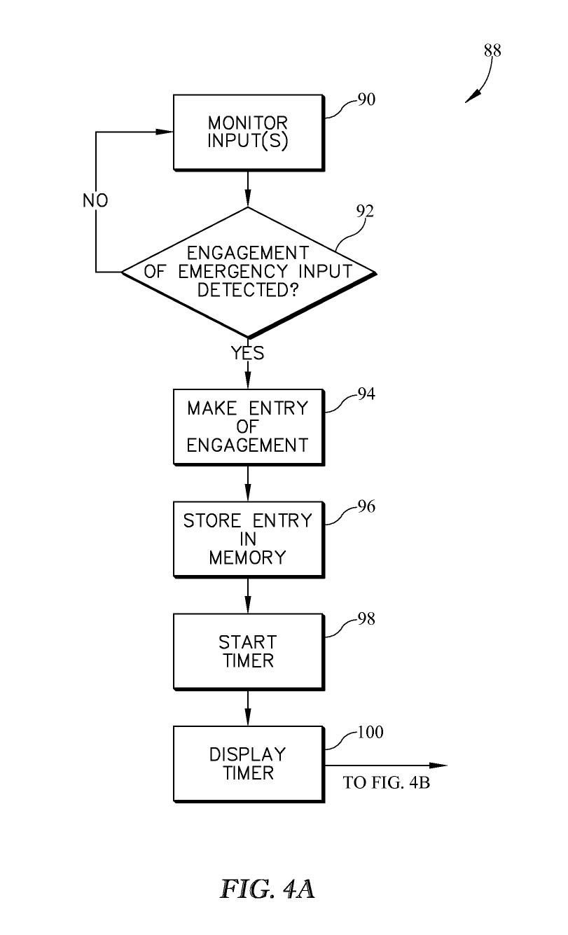

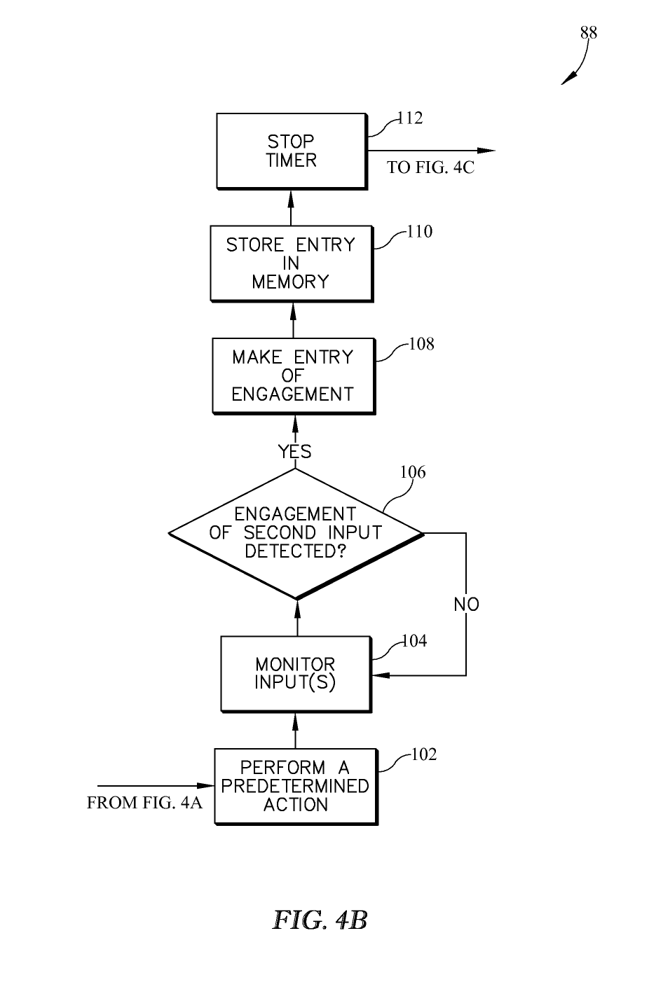

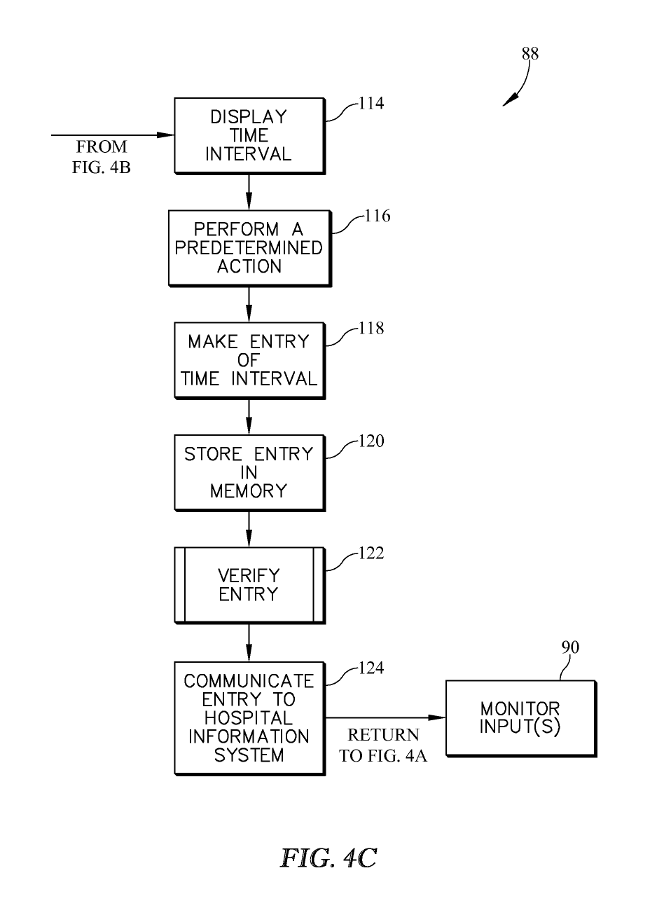

Referring to FIG. 4A-4C, a set of controller instructions 88 are shown that define a sequence of steps in which the engagement of one of the emergency switches and the engagement of the second input 51 result in the entry made by the controller 11 in the patient's electronic history file. The set of controller instructions 88 may be stored in the memory 15 included in the controller 11 and executed by the processor 13 included in the controller 11 to ensure that the time interval calculated by the stat clock 18 and entered into the patient's electronic history file is correct to provide evidence of compliance with the hospital protocol for administering care and delivering medical treatment.

Referring to FIG. 4A, the set of controller instructions 88 begins with a first monitor input step 90 that initializes the step sequence. The first monitor input step 90 directs the controller 11 to monitor the emergency inputs associated with the bed 10 such as the CPR handle 44, the Trendelenburg pedal 46, and the reverse Trendelenburg pedal 48 for signals indicating that any one or more of the emergency inputs is engaged.

The set of controller instructions 88 further includes an engagement detected query 92 that is executed by the controller 11 following the first monitor input step 90 as shown in FIG. 4A. The engagement detected query 92 directs the controller 11 to determine whether a signal indicating the engagement or enablement of any one of the emergency inputs is detected. In response to a NO answer being provided to the engagement detected query 92, the controller 11 returns to the first monitor input step 90.

The set of controller instructions 88 further includes a make entry step 94 that is executed by the controller 11 in response to a YES answer being provided to the query 92 as shown in FIG. 4A. The make entry step 94 directs the controller 11 to make an entry indicating that one ore more emergency inputs were detected. The entry may also include the time and date at which the engagement of the one or more emergency inputs were detected.

The set of controller instructions 88 further includes a store entry step 96 that is executed by the controller 11 following the make entry step 94 as shown in FIG. 4A. The store entry step 96 directs the controller 11 to store the entry made in the make entry step 94 in the memory 15 of the controller 11 in preparation for communicating the entry to the medical records database 24.

The set of controller instructions 88 further includes a start timer step 98 that is executed by the controller 11 following the first store entry step 96 as shown in FIG. 4A. The start timer step 98 directs the controller 11 to initialize the stat clock 18 to measure the time elapsing following the detected engagement of the one or more emergency inputs. The start timer step 98 may coincide with the real-time clock 16 being displayed on the display 52 of the user interface 14 as suggested in FIG. 4A. The real-time clock 16 may indicate the time and date at which the engagement of the one or more emergency inputs is detected using the emergency start designator shown in FIG. 6.

The set of controller instructions 88 further includes a display timer step 100 that is executed by the controller 11 following the start timer step 98 as shown in FIG. 4A. The display timer step 100 directs the controller 11 to display the stat clock 18 on the display 52 of the user interface 14. The display timer step 100 may coincide with the real-time clock 16 being displayed on the display 52 of the user interface 14 at the same time the stat clock 18 is displayed.

Referring to FIG. 4B, the set of controller instructions 88 further includes a perform action step 102 that is executed by the controller following the display timer step 100 as shown in FIG. 4B. The perform action step 102 directs hospital personnel to perform a first predetermined action associated with the detected engagement of the one or more emergency inputs, such as activating the room light 58 or any one or more of the operator tasks 80 shown in FIG. 3. The first perform action step 102 may coincide with a first action prompt issued by the controller 11 to the user interface 14. The first action prompt may provide an audible or visual indication to hospital personnel to perform the predetermined action(s).

The set of controller instructions 88 further includes a monitor input step 104 that is executed by the controller 11 following the first perform action step 102 as shown in FIG. 4B. The second monitor input step 104 directs the controller 11 to monitor the second input 51 for a signal indicating that the second input 51 is engaged or enabled. As mentioned above, the second input 51 may be displayed as a button on the display 52 of the user interface 14 following the detected engagement of the one or more emergency inputs. The second input 51 may be enabled or engaged by hospital personnel located within the patient's room 50.

The set of controller instructions 88 further includes an engagement detected query 106 that is executed by the controller 11 following the second monitor input step 104 as shown in FIG. 4B. The second engagement detected query 106 directs the controller 11 to determine whether a signal indicating the engagement or enablement of the second input 51 is detected. In response to a NO answer being provided to the second engagement detected query 106, the controller 11 returns to the monitor input step 104 as shown in FIG. 4B.

The set of controller instructions 88 further includes a make entry step 108 that is executed by the controller 11 in response to a YES answer being provided to the engagement detected query 106 as shown in FIG. 4A. The make entry step 108 directs the controller 11 to update the entry made in the first make entry step 94 to indicate that the second input 51 was detected. The make entry step 108 may also direct the controller to add the date and time in which the engagement of the second input 51 is detected.

The set of controller instructions 88 further includes a store entry step 110 that is executed by the controller following the make entry step 108 as shown in FIG. 4B. The store entry step 110 directs the controller 11 to store the updated entry made in the second make entry step 108 in the memory 15 of the controller 11 in preparation for communicating the updated entry to the nurse call master station 56.

The set of controller instructions 88 further includes a stop timer step 112 that is executed by the controller 11 following the store entry step 110 as shown in FIG. 4B. The stop timer step 112 directs the controller 11 to stop the stat clock 18 in response to the detected engagement of the second input 51 to signify the end of the emergency condition(s) associated with the engagement of the one or more emergency inputs. The real-time clock 16 may indicate the time and date at which the engagement of the second input 51 was detected by the controller 11 in the emergency end designator shown in FIG. 6. The stop timer step 112 may coincide with the use of stat clock 18 to calculate the time interval defined by the start timer step 98 and the stop timer step 112. The calculated time interval may be temporarily stored in the memory 15 of the controller 11 in preparation for communicating the time interval to user interface 14 in a display time interval step 114 as shown on FIG. 4C.

Referring to FIG. 4C, the set of controller instructions 88 further includes the display time interval step 114 that is executed by the controller 11 following the stop timer step 112. The display time interval step 114 directs the controller 11 to display the time interval calculated by the stat clock 18 on the display 52 of the user interface 14 as shown in FIG. 6.

The set of controller instructions 88 further includes a perform action step 116 that is executed by the controller 11 following the display time interval step 114 as shown in FIG. 4C. The perform action step 116 directs hospital personnel to perform a predetermined action in response to the end of the emergency condition defined by stop timer step 112. The second predetermined action may include de-activating the room light 58, de-activating the emergency light 60 activated during the activate emergency light task 86, notifying hospital personnel that the emergency condition identified in the alert caregivers task 84 has ended, or updating the patient's record in the update patient records task 82. The second perform action step 116 may coincide with a second action prompt issued by the controller 11 to the user interface 14. The second action prompt may provide an audible or visual indication to hospital personnel to perform the second predetermined action(s).

The set of controller instructions 88 further includes a make entry step 118 that is executed by the controller 11 following the perform action step 116 as shown in FIG. 4C. The make entry step 118 directs the controller 11 to update the entry made in the make entry step 108 to include the time interval calculated by the stat clock 18 and displayed on the display 52 during the display time interval step 114.

The set of controller instructions 88 further includes a store entry step 120 that is executed by the controller 11 following the make entry step 118 as shown in FIG. 4C. The store entry step 120 directs the controller 11 to store the updated entry made in the make entry step 118 in the memory 15 of the controller 11 in preparation for communicating the updated entry to the medical records database 24.

The set of controller instructions 88 further includes verify entry step 122 that is executed by the controller 11 following the store entry step 120 as shown in FIG. 4C. The verify entry step 122 directs the controller 11 to obtain an affirmation from hospital personnel that the entry stored in the store entry step 120 by the controller 11 is correct prior to communicating the entry to the medical records database 24. The verify entry step 122 may include an authorization sub-step accompanied by a first authorize notification that is issued by the controller 11 to the user interface 14. The first authorize notification may be a visual or audible indication to hospital personnel to provide an electronic signature indicating the correctness of the patient entry stored in the store entry step 120. In other embodiments, the authorization sub-step may be accompanied by a second authorize notification that is issued by the controller 11 to one or more of the caregiver alert devices 62 through the healthcare communication system 20. The second authorize notification may be a visual or audible indication to hospital personnel to remotely provide the electronic signature indicating the correctness of the patient entry stored in the third store entry step 120.

The set of controller instructions 88 further includes a communicate entry step 124 that is executed by the controller 11 following the verify entry step 122 as shown in FIG. 4C. The communicate entry step 124 directs the controller 11 to communicate the entry stored in the third store entry step 120 to the hospital information system, also referred to as the nurse call master station 56. The entry may then be incorporated into the patient's electronic history file to provide evidence of adherence to the hospital protocol for delivering medical care and administering treatment. The controller 11 returns to the first monitor input step 90 following the execution of the communicate entry step 124 as shown in FIG. 4C.

1. A patient support apparatus comprising
a frame movable between an operating position and a first emergency position,
a first emergency input indicative of an emergency condition, the first emergency input being coupled to the frame to cause the frame to move toward the first emergency position in response to engagement of the first emergency input, and
a controller coupled to the first emergency input to detect engagement of the first emergency input and make an entry into a patient's electronic medical record of the engagement of the first emergency input.

2. The patient support apparatus of claim 1, wherein the controller includes memory and the controller stores the entry in the memory.

3. The patient support apparatus of claim 2, wherein the controller is configured to transmit the entry stored in the memory via a communication network to an electronic database for inclusion in the patient's electronic medical record.

4. The patient support apparatus of claim 1, further comprising a second input indicative of an end to the emergency condition and the controller is further configured to detect engagement of the second input and add to the entry engagement of the second input.

5. The patient support apparatus of claim 1, further comprising a graphical user interface coupled to the frame and the controller is coupled to the graphical user interface to cause the graphical user interface to display a visual indicator communicating that the emergency condition is occurring in response to engagement of the first emergency input.

6. The patient support apparatus of claim 5, further comprising an emergency light coupled to the controller and configured to provide light in response to engagement of the first emergency input.

7. The patient support apparatus of claim 6, wherein the patient support apparatus is located in a patient room and the emergency light is located outside the patient room.

8. The patient support apparatus of claim 1, wherein the first emergency input is a CPR handle coupled to the frame to move relative to the frame between an engaged position which causes the frame to move toward the first emergency position and a disengaged position which allows the frame to remain in the operating position and the first emergency position is a CPR position.

9. A patient support apparatus comprising
a frame movable between an operating position and a first emergency position,
a first emergency input indicative of an emergency condition, the first emergency input being coupled to the frame to cause the frame to move toward the emergency position in response to engagement of the first emergency input, and
a controller coupled to the first emergency input to detect engagement of the first emergency input and start a timer upon engagement of the first emergency input.

10. The patient support apparatus of claim 9, wherein the controller is further configured to make an entry into a patient's medical record of the engagement of the first emergency input.

11. The patient support apparatus of claim 10, wherein the controller is further configured to communicate the entry via a communication network to an electronic database for inclusion in a patient's electronic medical record.

12. The patient support apparatus of claim 9, further comprising a graphical user interface and the controller is coupled to the graphical user interface to cause the timer to be displayed upon engagement of the first emergency input.

13. The patient support apparatus of claim 12, wherein the controller is further configured to cause a real-time clock to be displayed on the graphical user interface at the same time the timer is displayed.

14. The patient support apparatus of claim 9, further comprising a second input indicative of an end to the emergency condition and the controller is further configured to detect engagement of the second input and add to the entry engagement of the second input.

15. The patient support apparatus of claim 14, wherein the controller is further configured calculate a time interval in response to detecting the second input and add the time interval to the entry.

16. The patient support apparatus of claim 15, wherein the controller is further configured to communicate the entry via a communication network to an electronic database for inclusion in a patient's electronic medical record.

17. The patient support apparatus of claim 14, wherein the second input is detected by the controller in response to movement of the frame from the first emergency position to the operating position.

18. The patient support apparatus of claim 17, wherein the controller is further configured calculate a time interval in response to detecting the second input, add the time interval to the entry, and communicate the entry via a communication network to an electronic database for inclusion in a patient's electronic medical record.

19. The patient support apparatus of claim 9, wherein the first emergency input is a CPR handle coupled to the frame to move relative to the frame between an engaged position which causes the frame to move toward the first emergency position and a disengaged position which allows the frame to remain in the operating position and the first emergency position is a CPR position.

20. The patient support apparatus of claim 9, wherein the controller is further configured to send a priority nurse call to a hospital information system via a communication network in response to engagement of the first emergency input.
